(19) United States
(12) Patent Application Publication
(10) Pub. No.: US 2025/0331870 A1
(43) Pub. Date: Oct. 30, 2025

(54) SURGICAL GUIDE FOR OSTEOSYNTHESIS SURGERY IN PARTICULAR OF THE HALLUX VALGUS

(71) Applicant: NOVASTEP, Rennes (FR)

(72) Inventors: Brian SCHAUER, Huntersville, NC (US); Thomas CHEVREL, Rennes (FR); Loïc GIROD, Goven (FR); Chloé LAROCHE, Rennes (FR)

(21) Appl. No.: 19/192,978

(22) Filed: Apr. 29, 2025

Related U.S. Application Data
(60) Provisional application No. 63/640,268, filed on Apr. 30, 2024.

(51) Int. Cl. A61B 17/17; A61B 17/00; A61F 2/42; A61F 2/46

(52) U.S. Cl. CPC A61B 17/1775; A61B 17/1703; A61F 2/4225; A61F 2/4606; A61B 2017/0092; A61F 2002/4677; A61F 2002/4687

(57) ABSTRACT

A surgical guide for osteosynthesis surgery is disclosed. The surgical guide includes a guide body comprising a head and a base, the head extending from one end of the base. The surgical guide also includes a guiding portion allowing the surgical guide to be positioned on a targeting wire inserted into a bone and a locking device configured to secure the surgical guide on the targeting wire when the surgical guide is positioned on the targeting wire. The surgical guide also includes a targeting arm adapted to cooperate with the base of the guide body so as to be displaceable relative to the guide body, the targeting arm having at a distal end thereof at least one opening for supporting the at least one guiding wire.

### CROSS REFERENCE TO RELATED APPLICATIONS

The present application claims priority to and the benefit of U.S. provisional application No. 63/640,268, filed Apr. 30, 2024, which is hereby incorporated by reference herein in its entirety.

### FIELD

The present invention concerns the field of surgical guides and more particularly a surgical guide for osteosynthesis surgery of the hallux valgus, in particular, in mini-invasive or percutaneous approach such as bunion osteotomy surgery. By mini-invasive approach, it should be understood a surgery limiting the approach accesses to a few centimeters and by percutaneous approach, it should be understood a surgery limiting the approach accesses to a few millimeters.

### BACKGROUND

The deformation of the hallux valgus results from a cuneiform-metatarsal hypermobility upon a metatarsus varus. The metatarsus varus is accentuated at the bearing phase. There is then observed an inward deviation, called varus of the first metatarsal, whereas the first phalanx, retained by its joint capsule and the sesamoidal line, deviates outwards, which is called valgus.

There are different surgical techniques allowing correcting this deformation.

In the context of a mini-invasive or percutaneous approach surgery in the correction of the hallux valgus, one amongst the commonly used techniques is called percutaneous bunion correction, which consists of cutting the first metatarsal into two bone fragments, translating the first metatarsal's head (one of the bone fragments) and fusing the two bones fragments by means of one or two screws.

During the surgical procedure, the translation of the first metatarsal's head and its stabilization are the most decisive and the most complex steps to perform.

The correct position and orientation of the first metatarsal's head will condition the anatomical congruence of the bone fragments during the insertion of the screws that will support the osteosynthesis. This position is also critical to restore the mobility of the metatarso-phalangeal joint and then the biomechanical function of the hallux.

The procedure is often performed freehand, usually including adjustments of the position of guiding wires used to help the final implant insertion (screws) and is therefore subject to a high level of uncertainty at the time of preparation and usually requires a long adjustment phase.

Usually, the steps of the first metatarsal's head translation and guiding wire insertion are done under fluoroscopy. This results in numerous of X-ray images, which can be detrimental to the health of both patients and operators. In addition, the bone surfaces are not always congruent, and the metatarsal is not correctly reduced, resulting in poor implant positioning, possible mechanical weakness of the system, and therefore failure of the operation.

In order to remedy these drawbacks, several systems have been developed to guide the insertion of the guide wires in the first metatarsal and to control the translation of the first metatarsal's head.

### SUMMARY

Hence, one amongst the objects of the invention is to provide an improved surgical guide for osteosynthesis surgery for percutaneous bunion osteotomy surgery of simple design and intuitive use, offering the advantage of requiring only small incisions, guiding the insertion of implants in a reproducible manner while limiting the number of X-ray images and providing high stability for the surgeon.

To this end, and in accordance with the invention, there is provided a surgical guide for percutaneous bunion osteotomy surgery as well as a new surgical technique, allowing the insertion into bone fragments of guiding wires guiding the definitive implants before performing the osteotomy cut.

To this end, and in accordance with the invention, there is provided a surgical guide for osteosynthesis surgery, for example forming an aimer for guiding and inserting at least one guiding wire into a bone, comprising:
a guide body comprising a head and a base, the head extending at one end of the base of the guide body,
a guiding portion allowing the surgical guide to be positioned with respect to, for example on, a targeting wire inserted into a bone,
a locking device configured to secure the surgical guide with respect to, for example on, the targeting wire, when the surgical guide is positioned with respect to, for example on, on the targeting wire.
a targeting arm adapted to cooperate with the base of the guide body so as to be displaceable relative to the guide body, the targeting arm having at a distal end thereof at least one opening for supporting the at least one guiding wire.

The securing of the surgical guide on the targeting wire allows to provide a huge stability for placing guiding wires into the bone.

This allows the practitioner to place guiding wires into the bone without holding the surgical guide. This is also effective as with the invention, the practitioner places guiding wires before performing the cut and the translation of the metatarsal head which improves the stability of the full construct.

Advantageously, the guiding portion includes a through hole formed within the head of the guide body and a sleeve provided with a through hole, the sleeve extending from the head so that the through hole of the sleeve is aligned with the through hole of the head.

Advantageously, the locking device comprises a clamping knob comprising a threaded portion, and a threaded hole formed within the head of the guide body allowing to screw the clamping knob so as to secure the surgical guide to the targeting wire.

Advantageously, the surgical guide comprises an external visualization device configured to allow a preview of the at least one guiding wire insertion direction, the external visualization device comprising connecting means configured to connect the external visualization device to the targeting arm.

Advantageously, the external visualization device comprises a first channel configured to hold the at least one guiding wire, wherein the connecting means comprise a first guiding pin receivable in the hole of the targeting arm, wherein the first channel and the first guiding pin are arranged on a same plane, with an offset distance between the first channel and the first guiding pin.

Advantageously, the surgical guide comprises a monoaxial guiding device configured to hold a single guiding wire, the monoaxial guiding device comprising a single hole to hold the single guiding wire and further comprising connecting means to connect the monoaxial guiding device to the targeting arm.

Advantageously, the guide body and the targeting arm are made of a radiolucent material. This offers the ability to inspect the proper direction of the guiding wires under X-ray without the visualization being obscured by any radiopaque material of the instrumentation.

According to an aspect, the invention concerns an assembly comprising the surgical guide as previously described and a targeting wire receivable through the hole of the head of the guide body.

Advantageously, the targeting wire comprises a first part configured to be inserted into the bone and a second part configured to hold the surgical guide, wherein the first part comprises a threaded portion.

Advantageously, the targeting wire comprises a physical stop configured to prevent the surgical guide from moving beyond the second part of the targeting wire.

According to an aspect, the invention concerns a method comprising:
Inserting a targeting wire in a bone;
Positioning a surgical guide as previously described on the targeting wire;
Inserting at least one guiding wire in the bone using the surgical guide;
Performing a cut of the bone to form a first part of the bone and a second part of the bone; the at least one guiding wire being for example inserted into the second part of the bone.

Translating the first part of the bone with respect to the second part of the bone Screwing at least one fixation screw into the first and second parts of the bone by using the at least one guiding wire.

By placing the guiding wires before performing the cut and the translation of the metatarsal head the stability of the full construct is improved.

Advantageously, the method comprises fixing the surgical guide on the targeting wire, before inserting the at least one guiding wire in the bone using the surgical guide.

Advantageously, fixing the surgical guide on the targeting wire comprises screwing a clamping knob into a threaded hole of the surgical guide until a tip of the clamping knob comes into contact with the targeting wire.

Advantageously, the method comprises removing the surgical guide from the targeting wire and removing the targeting wire from the bone, before performing the cut of the bone.

Advantageously, performing the cut of the bone comprises inserting a cutting device along the direction of insertion of the targeting wire.

Advantageously, the method comprises previewing, by means of an external visualization device, the insertion direction of the at least one guiding wire, before inserting the at least one guiding wire in the bone.

### BRIEF DESCRIPTION OF THE DRAWINGS

Other advantages and features will appear better from the following description of several variants, provided as non-limiting examples, of the surgical guide in accordance with the invention, with reference to the appended drawings wherein:

FIG. 1 shows an assembly comprising a surgical guide according to the invention,

FIG. 2 shows a targeting wire of the assembly of the invention,

FIG. 3 shows a guide body of the surgical guide of the invention,

FIG. 4 shows a targeting arm of the surgical guide of the invention,

FIG. 5 shows a first clamping knob of the surgical guide of the invention,

FIG. 6 shows a second clamping knob of the surgical guide of the invention,

FIG. 7 shows a first sleeve of the assembly of the invention,

FIG. 8 shows a first guiding wire of the assembly of the invention,

FIG. 9 shows a monoaxial guiding device of the surgical guide of the invention,

FIG. 10a shows an external visualization device of the surgical guide of the invention,

FIG. 10b shows the external visualization device of the surgical guide according to one variant of the invention,

FIGS. 11 to 30 show the surgical guide according to the invention at the different steps of use of said surgical guide.

### DETAILED DISCLOSURE

In the following description of the surgical guide according to the invention, the same reference numerals refer to the same elements. The different views are not necessarily plotted to scale. Moreover, the surgical guide according to the invention is particularly suited to an osteosynthesis for correction of a hallux valgus deformity but it is quite obvious that it could be suited to the osteosynthesis of any other portion of the anatomy of the human body yet without departing from the scope of the invention.

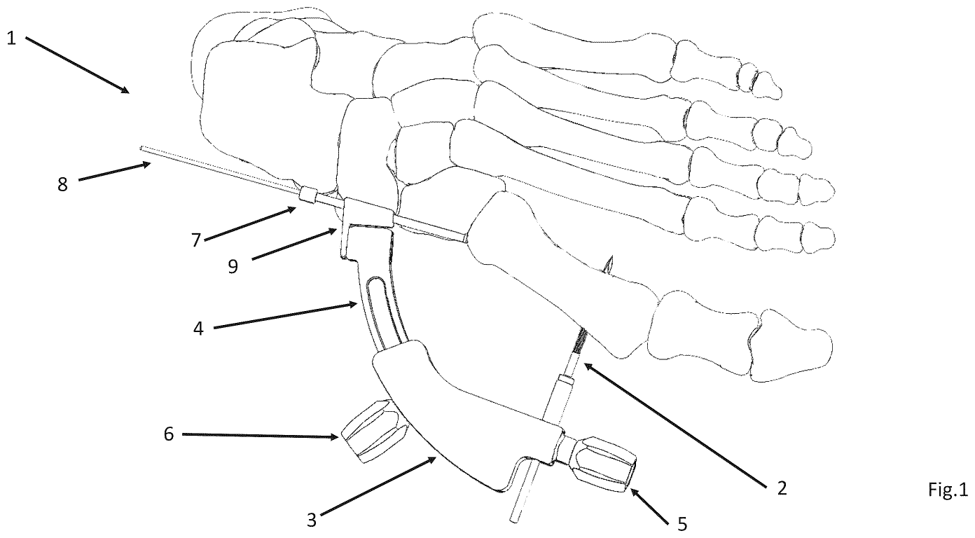

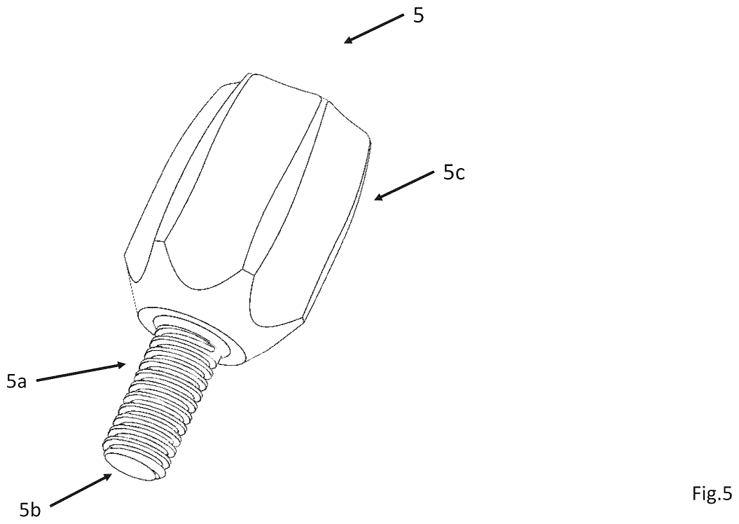

Referring to FIGS. 1 to 10, an assembly according to the invention comprises a surgical guide 1 comprising a guide body 3, the guide body 3 comprising a head 30 and a base 32, the head extending from one end of the base 32, the surgical guide 1 comprising a guiding portion allowing the surgical guide 1 to be positioned with respect to, for example on, a targeting wire 2 inserted into a bone, a locking device configured to secure the surgical guide 1 with respect to, for example on, the targeting wire 2, for example when the surgical guide is positioned on the targeting wire, a targeting arm 4 adapted to cooperate with the base 32 of the guide body 3 so as to be displaceable relative to the guide body 3, the targeting arm 4 having at a distal end thereof at least one opening, for example at least one hole 4b for supporting the at least one guiding wire 8.

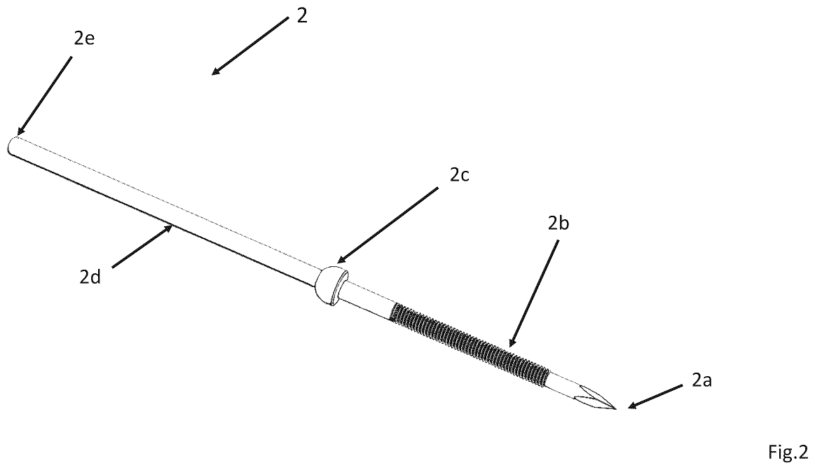

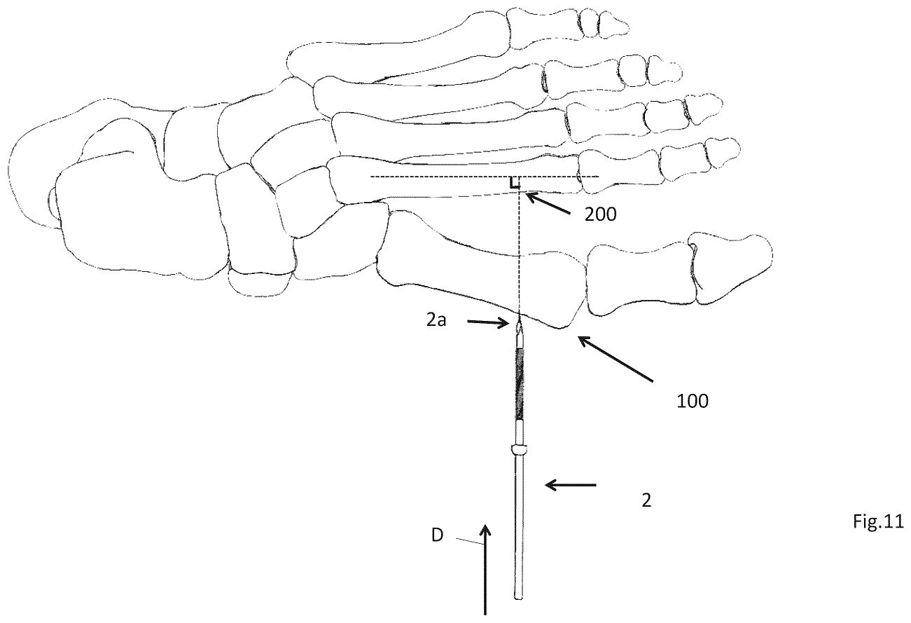

Referring to FIGS. 1 and 2, the assembly can comprise a targeting wire 2. The targeting wire 2 is configured to be inserted into a metatarsal bone so as to define a metatarsal head cut position (FIG. 11) and virtual final position of the metatarsal head. The targeting wire 2 is further configured to hold a guide body 3 of the surgical guide (FIG. 15).

As illustrated in FIG. 2, the targeting wire has a cylindrical shape. The targeting wire can comprise a first part intended to be inserted into the metatarsal bone and a second part intended to cooperate with the guide body 3 so as to hold the guide body 3.

The first part of the targeting wire 2 can comprise a trocar tip 2a, arranged for example at a first end of the targeting wire, allowing penetration of the targeting wire 2 into the bone and can comprise a threaded portion 2b, for example proximal to the trocar tip 2a, assuring anchorage of the targeting wire on the bone when the targeting wire has penetrated into the bone.

The second part of the targeting wire 2 can comprise a cylindrical portion 2d configured to cooperate with a hole of the guide body 3 so that the guide body 3 can be held by the targeting wire. The second part of the targeting wire 2 is configured to allow the guide body 3 to be displaced with respect to the targeting wire 2. The cylindrical portion 2d has an outer diameter adapted to be received in a hole of the guide body 3. The cylindrical portion 2d can extend from a physical stop 2c of the targeting wire 2 to a second end 2e of the targeting wire, opposite to the trocar tip 2a of the targeting wire 2. The second end of the targeting wire is configured to be inserted in the hole of the guide body 3. The targeting wire 2 is configured to guide the guide body 3 from the second end 2e of the targeting wire to the physical stop 2c of the targeting wire 2.

The physical stop 2c of the targeting wire is configured to prevent the guide body 3 to move beyond the second part, for example beyond the cylindrical portion 2d of the targeting wire 2, for example when the guide body 3 is positioned on the targeting wire 2. The physical stop 2c of the targeting wire is configured to prevent the guide body 3 to move to the first part of the targeting wire 2. The physical stop 2c is configured so that the guide body 3 can come in contact with the physical stop 2c. The physical stop 2c can be arranged between the first and second part of the guiding wire 2, for example at a middle part of the targeting wire 2 and can be formed in one single piece with the rest of the targeting wire 2. The physical stop 2c can further serve as a geometrical reference for the target of guiding wires 8 of the surgical guide 1.

The cylindrical portion 2d is further configured to allow insertion of the targeting wire 2 in the bone with a power tool.

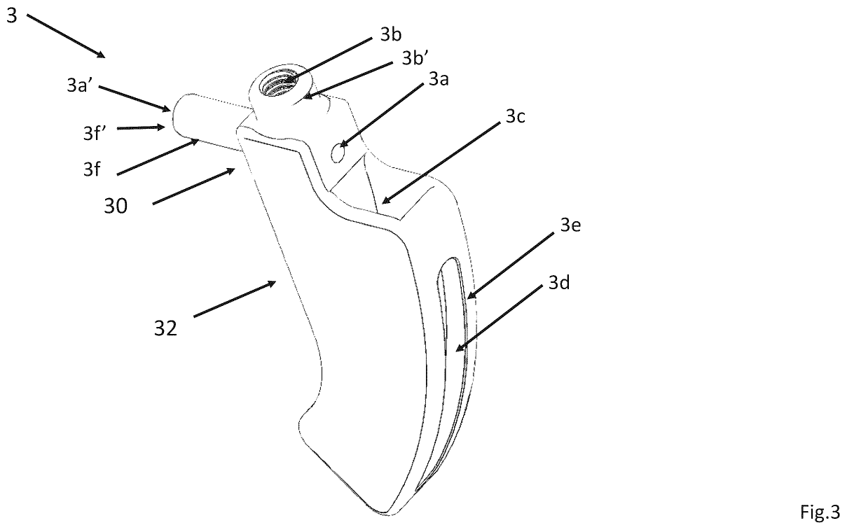

Referring to FIGS. 1 and 3, the surgical guide 1 can comprise a guide body 3. The guide body 3 can comprise a head 30 and a base 32, the head extending at an upper end of a base of the surgical guide, for example from an upper end of the base.

The head 3, which can have a substantially cube shape, is arranged on the upper base of the base and can be formed in one single piece with the base.

The guide body 3 can comprise a guiding portion configured to allow positioning of the guide body 3 on the targeting wire 2. For example the head 30 of the guide body 3, for example the middle part of the head 3 of the guide body, can be provided with a through hole 3a having a diameter adapted to receive at least a part of the second part of the targeting wire, for example the cylindrical portion 2d of the targeting wire. The guiding portion can further comprise an extension 3f of the head, forming a sleeve provided with a through hole having the same diameter as the diameter of the through hole 3a of the head, that is to say a diameter adapted to receive a part, for example another part of the cylindrical portion 2d of the targeting wire, for example another part of the cylindrical portion of the targeting wire. The extension 3f, which can have a tubular shape, can be arranged, with respect to the head so that the through hole 3a′ of the extension 3f is aligned with the through hole 3a of the head 30, for example in the continuity, so as to form a single hole 3a, 3a′ of the guiding portion having a diameter adapted to receive a part of the cylindrical portion 2d of the targeting wire. The extension 3f can be arranged on a lateral side of the head 30. The single hole opens on both lateral sides of the guide body 3. The extension 3f can be formed in one piece with the head 30. The extension 3f comprises an end 3f configured to cooperate with the physical stop 2c of the targeting wire when the guide body is positioned on the targeting wire 2.

The guiding portion is configured so that the guide body 3 can be mounted on the targeting wire 2 and removed from the targeting wire 2. The guiding portion is configured so that the guide body is movable relative to the targeting wire 2.

The surgical guide 1 can comprise a locking device configured to secure the guide body 3 on the targeting wire 2, for example, when the guide body 3 is positioned on the targeting wire. The locking device is configured to secure the guide body 3 so that the guide body 3 cannot move with respect to the targeting wire.

The locking device is configured to removably secured the guide body 3 on the targeting wire 2.

The locking device is configured to selectively secured the guide body 3 on the targeting wire 2.

For example, the locking device can comprise a first clamping knob 5 comprising a threaded portion 5a, a flat tip 5b and a turning portion 5c.

The locking device can further comprise a threaded hole 3b allowing to screw the clamping knob so as to secure the guide body 3 to the targeting wire 2. The threaded hole 3b can be formed within a second extension 3b′ of the head 30, extending for example from an upper end of the head 30 and formed for example in one single piece with the head 30 of the guide body 3.

The first clamping knob 5 allows to stabilize the guide body 3 on the cylindrical portion 2d of the targeting wire 2, for example by screwing the turning button 5 until the flat tip 5b of the turning button 5 comes in contact with the cylindrical portion 2d of the targeting wire 2. To this end, threaded hole 3b of the locking device can open on a hole 3a, 3b of the guiding portion, for example on the hole 3a of the head 30.

The first clamping knob 5 is configured so as to stabilize the guide body 3 in latero-medial direction and also in rotation around the main axis of the targeting wire 2.

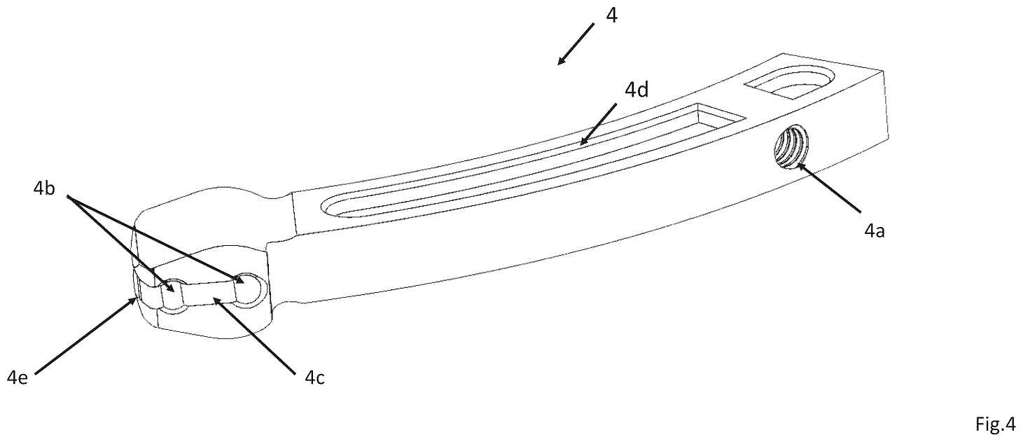

Referring to FIG. 3, the base 32 of the guide body 3 has a circular-arc shape whose concavity is directed towards the distal end 3f of the extension 3f. Said base 32 can be hollowed, the hollow forming a housing configured so as to receive at least a part of a targeting arm 4 (FIGS. 1 and 4). The housing can open on a lower end and the upper end of the base.

The housing can be configured so that the targeting arm can be movable relative to the guide body 3.

The surgical guide 1 can comprise a second locking device configured to removably secure the targeting arm 4 to the guide body 3. The locking device is configured for fixing the targeting arm 4 at a desired position.

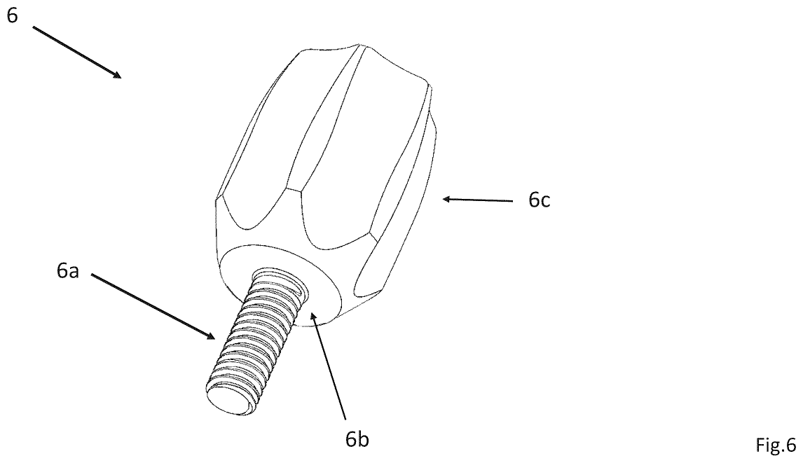

To this end, for example, the guide body 3 can be provided with a medial groove 3d, extending on a lateral side of the base 32, for example the medial external surface 3e of the base 32, and opening in the housing of the base 32. Said medial groove 3d is configured to guide a second clamping knob 6 (FIG. 6) intended to be attached to the targeting arm 4 so as to secure the targeting arm 4 to the guide body 3.

Referring to FIGS. 1 and 4, the surgical guide 1 can comprise the targeting arm 4. The targeting arm 4 is configured so as to be movable with respect to the guide body 3, for example the base 32 of the guide body 3. The targeting arm 4 has a circular-arc shape having the same radius of curvature as the base 32 of the guide body 3. Said targeting arm 4 has an aperture 4d extending substantially from the proximal end up to the distal end of the targeting arm 4.

The targeting arm 4 can be provided with a threaded hole 4a, for example arranged on a lateral side of the targeting arm 4 and configured to cooperate with the second clamping knob 6 of the surgical guide 1. For example, the threaded hole 4a can be configured so as to receive a threaded portion 6a of the second clamping knob 6.

Said second clamping knob 6 is shown on FIGS. 1 and 6. The surgical guide 1 can comprise the second clamping knob 6. The second clamping knob 6 can comprise a threaded portion 6a and a turning portion 6c. The turning portion 6c can comprises a end provided with a flat surface 6b, for example from which the threaded portion 6a extends.

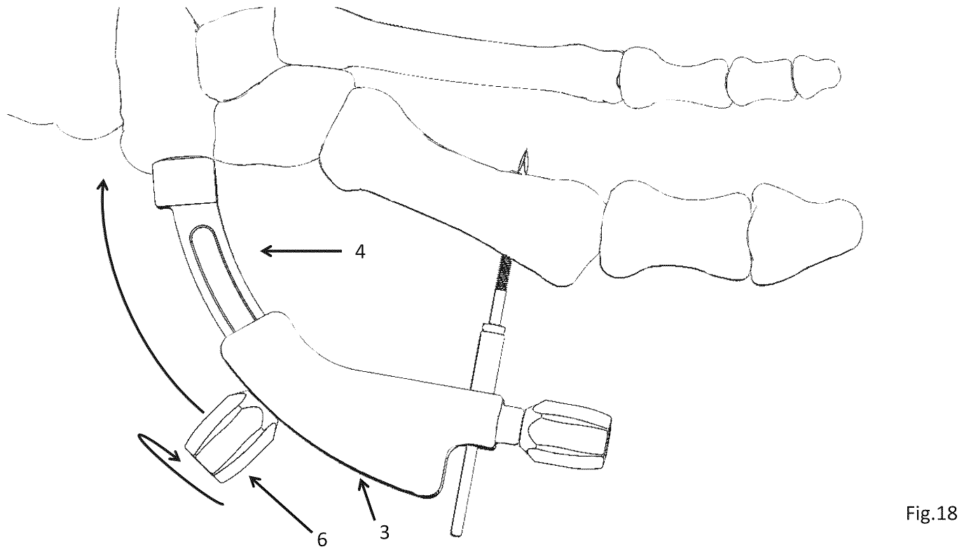

The second clamping knob 6 allows to block the targeting arm 4 at the desired position by screwing the second clamping knob 6 in the threaded hole 4a of the targeting arm until the flat surface 6b comes in contact with a medial external surface 3e of the base 32 of the guide body 3 (FIGS. 3 and 18).

Referring to FIG. 4 the targeting arm 4 can comprise, for example at its distal or lower end, tubular guiding holes 4b for holding sleeves. As this will be latter described, these guiding holes can be also configured to hold part of an external visualization device and part of a monoaxial guiding device.

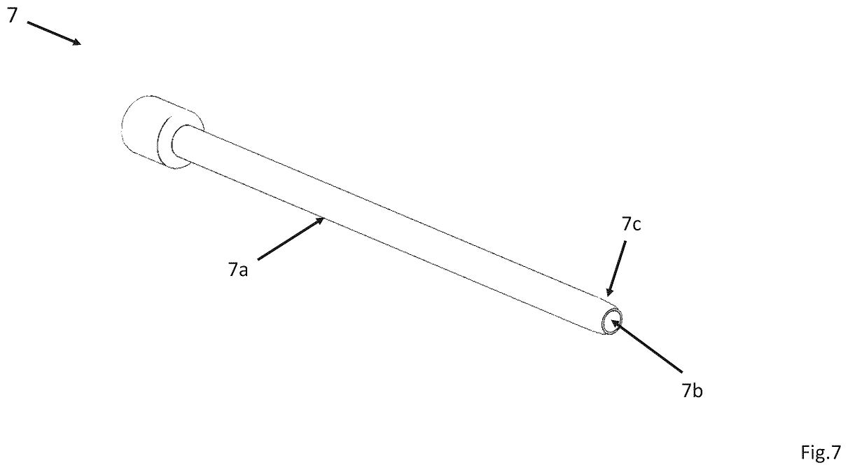

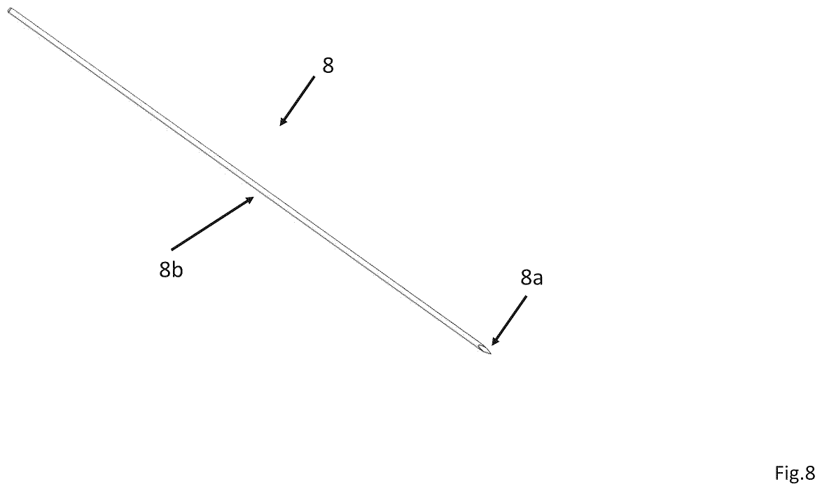

For example the targeting arm can comprise two guiding holes 4b for holding two sleeves (shown in FIG. 7). Each sleeve 7 may have a tubular body forming a channel configured to hold a guiding wire (shown in FIG. 8) ending with a tip 7c. Each sleeve can have an outer diameter 7a adapted to fit into the guiding holes 4b of the targeting arm 4. The sleeves 7 present an inner diameter 7b which fit with the guiding wire 8 diameter used. For example the inner diameter 7b of the sleeve 7 can be set up to 1.9 mm allowing to receive guiding wire provided with diameter 8 up to 1.8 mm.

Referring to FIGS. 1 and 8, the assembly can comprise two guiding wire each having an extended cylindrical portion 8b ending with a trocar tip 8a to ease penetration of the bone.

Advantageously, the targeting arm 4 may be provided with an internal groove 4c arranged between the two guiding holes 4b and opening on both guiding holes 4b so as to allow a passage of a guiding wire between the two guiding holes 4b. The targeting arm 4 can be further provided with a lateral groove 4e, opening on one of the two guiding holes 4b and on the outside of the targeting arm 4. The internal and lateral grooves allow to remove the targeting arm from the guiding wires, for example once the guiding wires have been inserted into the bones.

For example, the internal and lateral grooves 4c, 4e, width can be 1.9 mm allowing a passage and a use of all guiding wire with diameters up to 1.8 mm included.

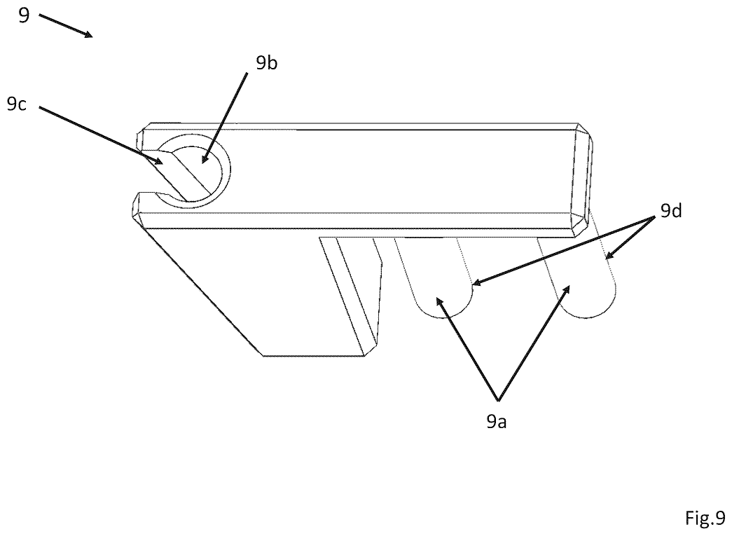

Referring to FIGS. 1 and 9 the surgical guide can comprise a monoaxial guiding device 9.

The monoaxial guiding device 9 is intended to be used for example when a single or unique guiding wire 8 need to be inserted into the bones.

The monoaxial guiding device 9 can comprise a single guiding hole 9b for holding a sleeve 7 as previously described, for example a single sleeve 7. The single guiding hole 9b extends along a direction parallel to the directions of the targeting arm guiding holes 4a, when the monoaxial guiding device 9 is connected to the targeting arm 4. The single guiding holes 9b and the targeting arm guiding holes 4a may be in a common plan.

The monoaxial guiding device 9 can comprise connecting means configured to adapt or connect the monoaxial guiding device 9 to the targeting arm 4. The connecting means can comprise two guiding pins 9a configured to be received in the guiding holes 4b of the targeting arm. The guiding pins have an outer diameter 9d adapted to fit into the guiding holes 4b of the targeting arm 4.

The monoaxial guiding device 9 can be provided with a lateral groove 9c, opening in the single guiding hole 9b and on the outside of the monoaxial guiding device 9. The lateral groove allows to remove the monoaxial guiding device 9 connected to the targeting arm 4 from the guiding wire, for example once the guiding wire have been inserted into the bones.

For example, lateral groove 9c can be 1.9 mm allowing a passage and a use of all guiding wire provided with diameters up to 1.8 mm included.

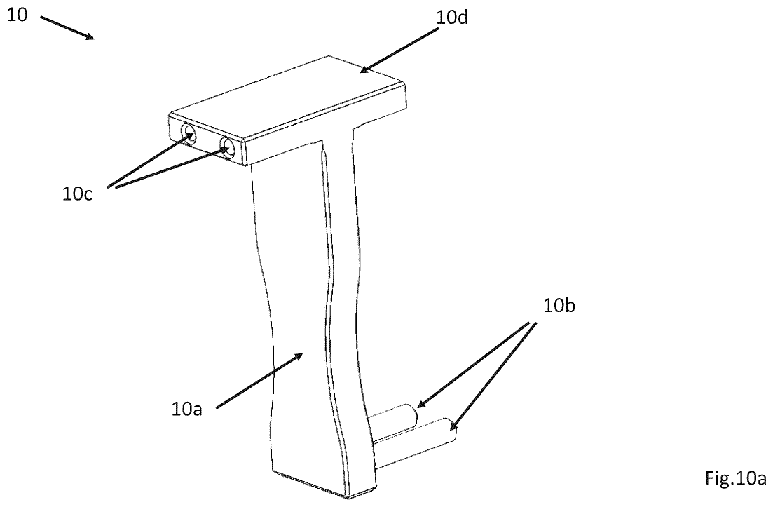

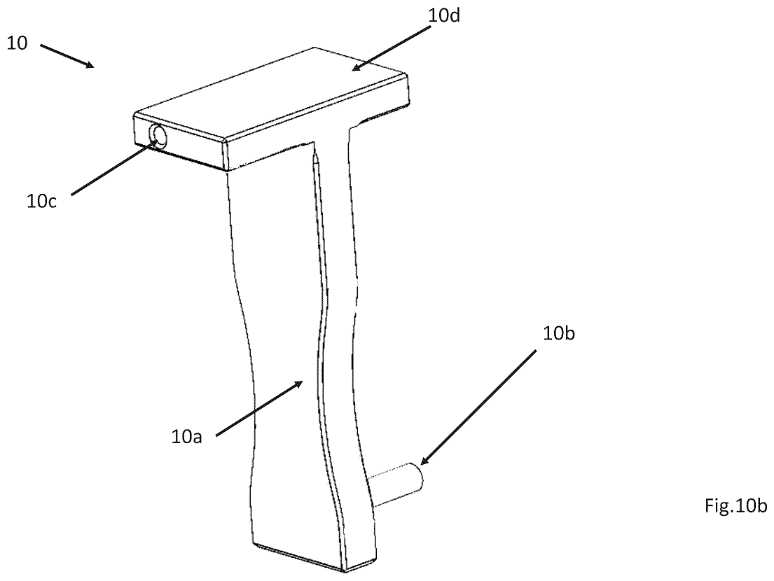

Referring to FIGS. 1 and 10, the surgical guide 1 can comprise an external visualization device 10. The external visualization device 10 allows to preview the insertion direction of the guiding wires 8. The external visualization device 10 is configured so as to externally visualize the proper direction of the guiding wires 8, for example before the guiding wires 8 are inserted into the bones.

As illustrated in FIG. 10a, the external visualization device 10 can comprise two channels 10c (medial and lateral channels) configured to hold the guiding wires 8. The channels present an inner diameter which fit with the guiding wire diameter 8 used. The channels are formed within a portion 10d of the external visualization device, which can extend from a plate 10a, for example orthogonally from a first end portion of the plate 10a of the external visualization device.

Alternatively, as illustrated in FIG. 10b, the external visualization device 10 can comprise a single or unique channel 10c configured to hold a guiding wire, for example when a single or unique guiding wire 8 need to be inserted into the bones. The external visualization device 10 allows to preview the insertion direction of the guiding wire 8. The external visualization device 10 is configured so as to externally visualize the proper direction of the guiding wire 8.

The external visualization device 10 can comprise connecting means configured to adapt or connect the external visualization device 10 to the targeting arm 4. The connecting means can comprise two guiding pins 10b (medial and lateral guiding pins) configured to be received in the guiding holes 4a of the targeting arm 4. The guiding pins 10b have an outer diameter adapted to fit into the guiding holes 4b of the targeting arm 4.

The guiding pins can 10b can extend from the plate of the external visualization device 10, for example orthogonally from the plate of the external visualization device, for example from an opposite second end portion of the external visualization device 10.

The medial channel main axis may be on the same plane as the medial guiding pin main axis, with an offset distance between these two axes. The lateral channel main axis may be on the same plane as the lateral guiding pin main axis, with an offset distance between these two axes.

When at least one guiding wire 8 is inserted into the at least one channel 10c of the external visualization device 10, said at least one guide wire 8 extends in a same direction than the one provided by a guiding wire 8 inserted into a guiding hole 4b of the targeting arm 4 by mean of a sleeve 7, with an offset.

Alternatively, when the external visualization device 10 comprises a single or unique channel 10c configured to hold a guiding wire, the channel main axis is arranged, for example with respect to the external visualization device so as to extend in the same direction as the guiding hole 9b of the monoaxial guiding device, when the monoaxial guiding device is connected to the targeting arm 4 (FIG. 10b).

The guide body 3 and the targeting arm 4 can be made of a radiolucent material (for example a plastic suitable for medical application). This offers the ability to inspect the proper direction of the guiding wires under X-ray without the visualization being obscured by any radiopaque material or instrumentation.

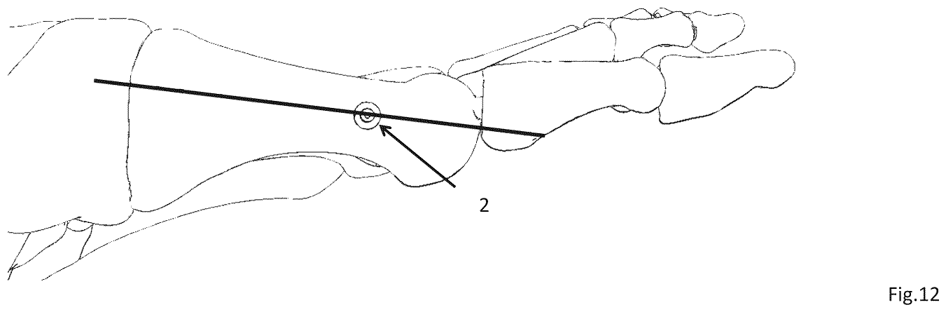

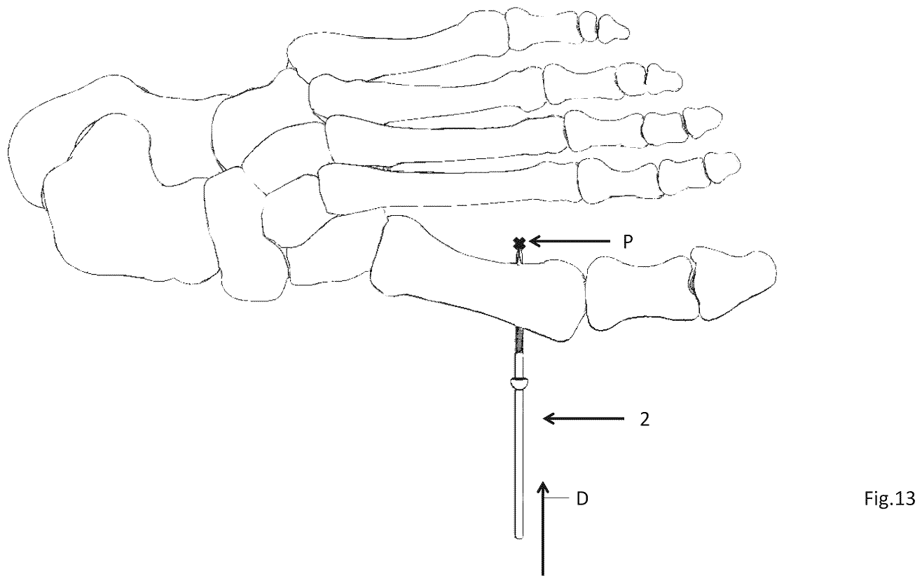

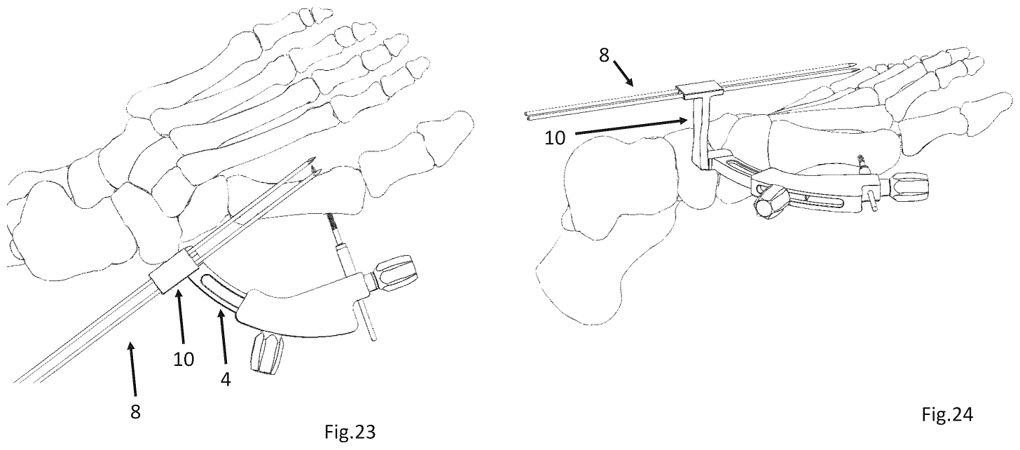

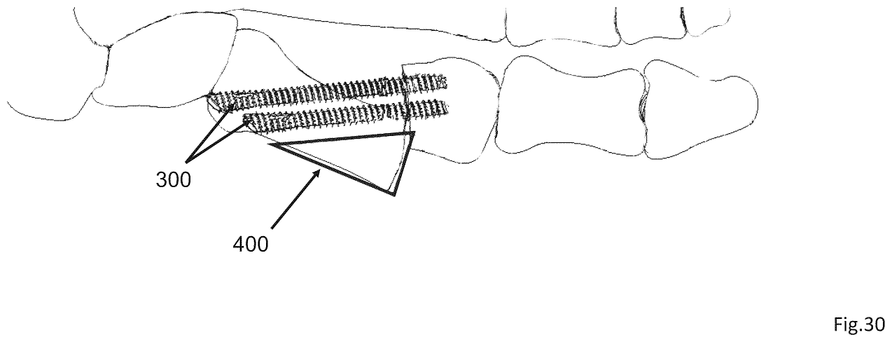

The operation of the surgical guide according to the invention will now be explained with reference to FIGS. 11 to 30 which illustrate a method for operating the previously described surgical guide. The surgical guide can be used for performing a bone correction and implant guidance to fuse two bone fragments, for example a percutaneous bunion correction.

Referring to FIGS. 11 to 13, the method comprises locating and inserting the targeting wire 2 in the medial area of the first metatarsal 100.

The method comprises for example Creating an incision of a few millimeters by medial approach using a scalpel, Inserting the trocar tip 2a of the targeting wire 2 in the middle of the sagittal plane of the first metatarsal 100, just below the metatarsal's head neck. Depending on the hallux valgus intermetatarsal angle, the targeting wire 2 may be directed perpendicular to the main axis of the first 100 or second metatarsal 200 (see arrow “D”).

Pursuing the insertion of the targeting wire 2 until the trocar tip 2a reaches the desired position (“P”) of the medial aspect of the metatarsal's head after correction. Once positioned, the targeting wire 2 is left in position to serve as a reference for the following steps of the procedure. The position and direction of the targeting wire 2 is the exact same as what will be the final osteotomy cut.

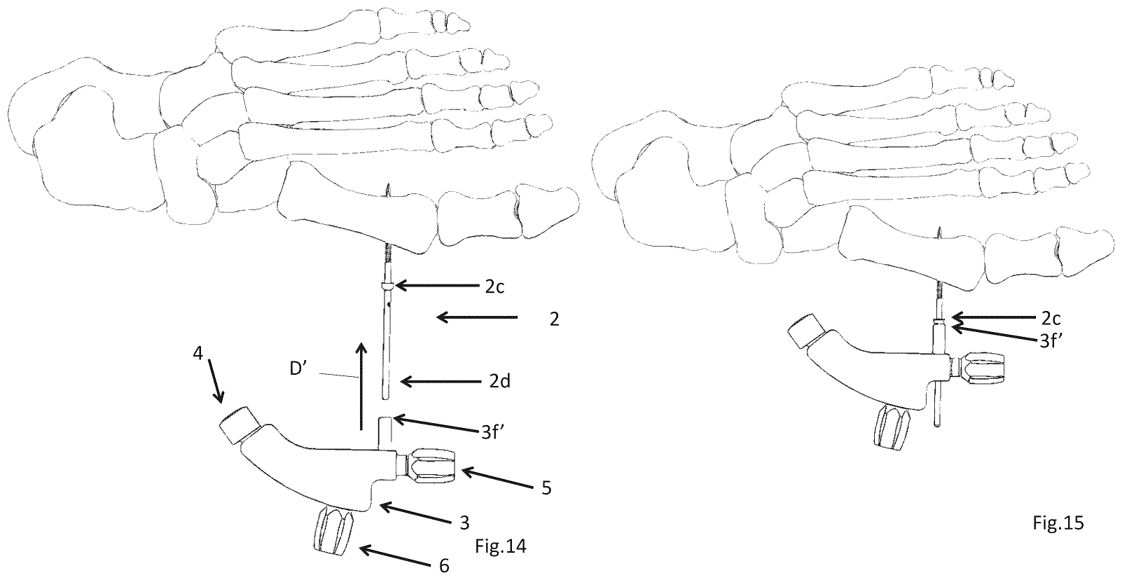

Referring to FIGS. 14 and 15, the next step consists in positioning the guide body 3 supporting the targeting arm 4, first clamping knob 5 and second clamping knob 6 over the targeting wire 2.

The method comprising for example Ensuring the initial position of the targeting wire 2 before placing the guide body 3, Positioning the guide body 3 on the targeting wire 2. The positioning comprising inserting (see arrow D′) the second part of the targeting wire in the hole of the guiding portion of the guide body 3 (FIG. 14) and moving the guide body 3 with respect to the targeting wire until the guide body 3, for example the extension end 3f touches the physical stop 2c of the targeting wire 2 (FIG. 15).

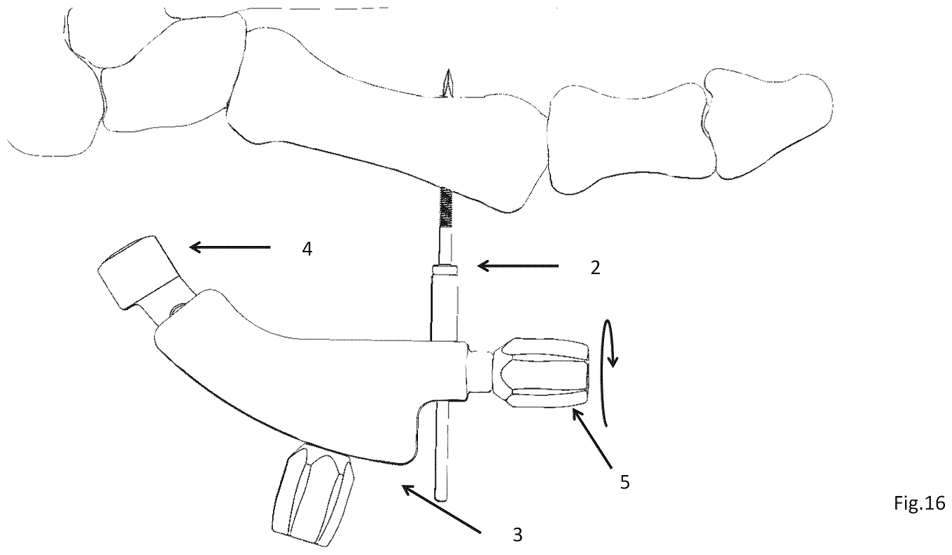

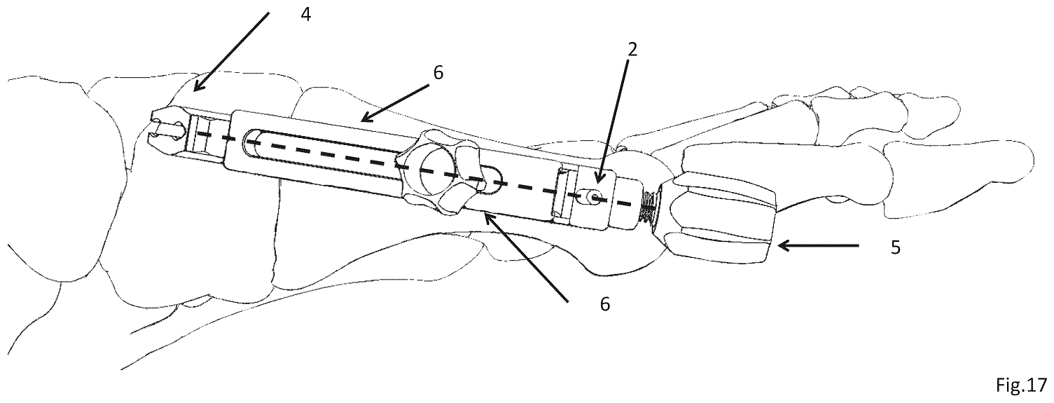

Referring to FIGS. 16 and 17, the method can comprise a next step of placing the guide body 3 in parallel position with the first metatarsal, and

Securing the guide body 3 to the targeting wire 2 by screwing clockwise the first clamping knob 5 until the guide body 3 is totally stabilized on the targeting wire 2.

Referring to FIG. 18, the method can comprise a next step of deploying the targeting arm 4 and adjusting the targeting arm position with the second clamping knob 6, the most proximal as possible to the metatarsal so as to best match the anatomy of the metatarsal, and

Locking the position of the targeting arm 4 by screwing clockwise the second clamping knob 6 until the targeting arm 4 is stabilized to the guide body 3.

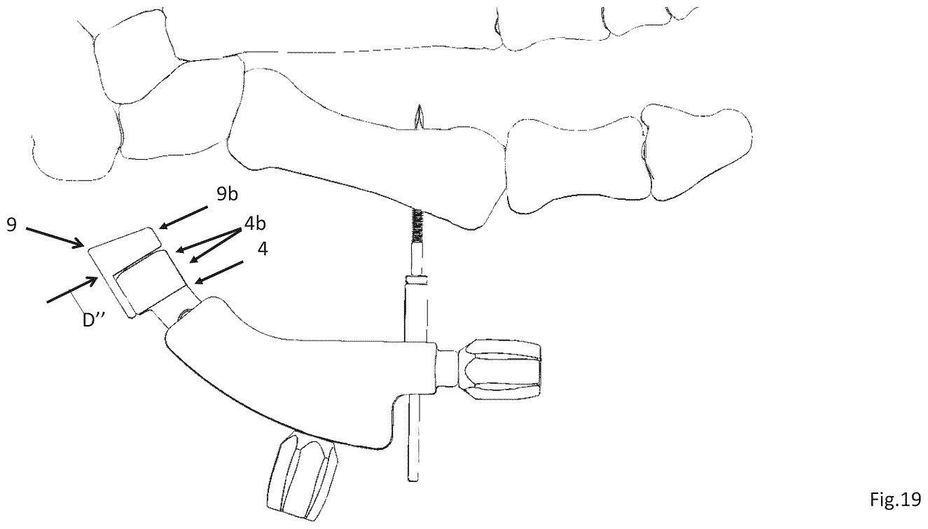

If a single screw is expected to fix the osteotomy or any other implant that would require a single guiding wire 8, the method can comprise a step of connecting the monoaxial guiding part 9 to the targeting arm 4 (see direction of insertion D″ in FIG. 19) by inserting the guiding pins 9a into the guiding holes 4b of the targeting arm 4.

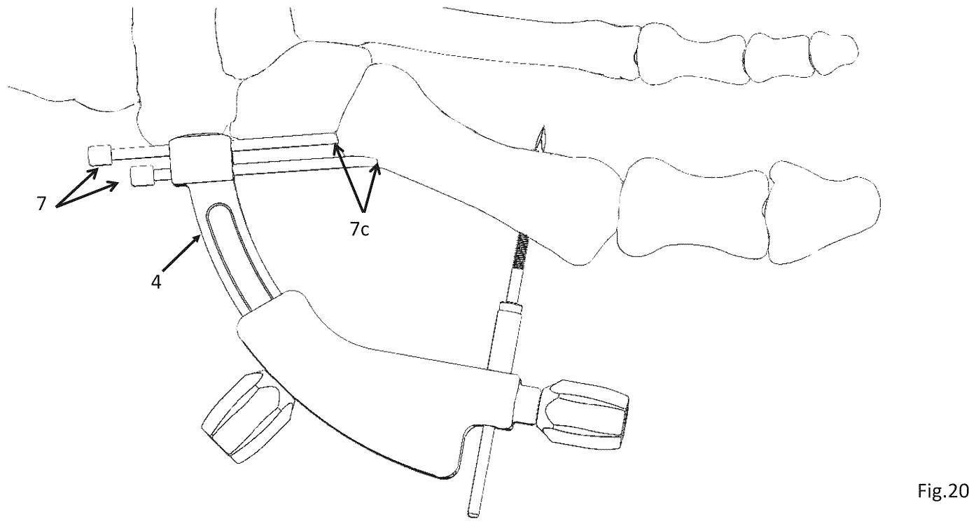

Referring to FIG. 20, the method can comprise a next step of inserting the two sleeves 7 or the single sleeve 7 into the guiding holes 4b of the targeting arm 4 or into the guiding hole 9b of the monoaxial guiding device 9, until the tip 7c of the sleeve 7 comes in contact with the bone.

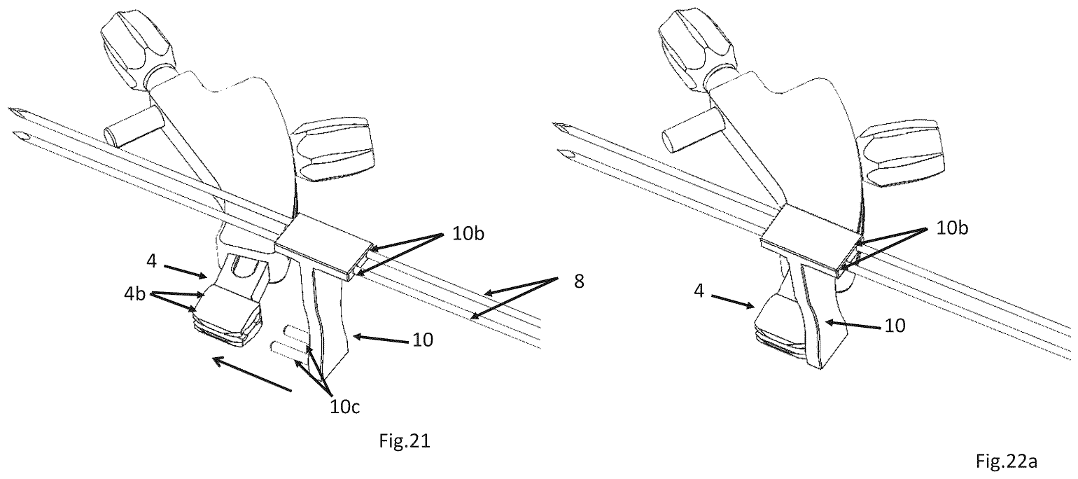

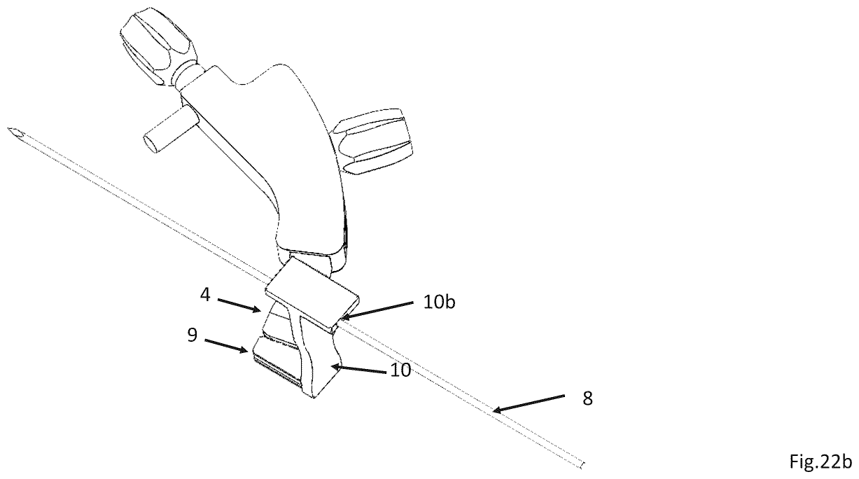

Referring to FIG. 21 to 24 in case the proper direction of the guiding wires 8 need to be checked before insertion in the bones, the method can comprise a step of connecting the external visualization device to the targeting arm 4 and then driving guiding wires 8 into the channels 10c of the external visualization device 10 (or the single guiding wire 8 into the single channel 10c (FIG. 22b). These wires 8 will be sagittaly offset from the main axis of the guiding holes 4b of the targeting arm 4, above the dorsal skin of the metatarsal, allowing the surgeon to externally visualize the proper trajectory of the guiding wires 8. This step can be performed before the step of inserting the two sleeves 7 into the guiding holes of the targeting arm 4. Once the proper direction has been checked, the external visualization device can be removed from the targeting arm 4, and the two sleeves 7 can be inserted into the guiding holes 4b of the targeting arm 4 (or the monoaxial guiding part 9 can be connected to the targeting arm 4).

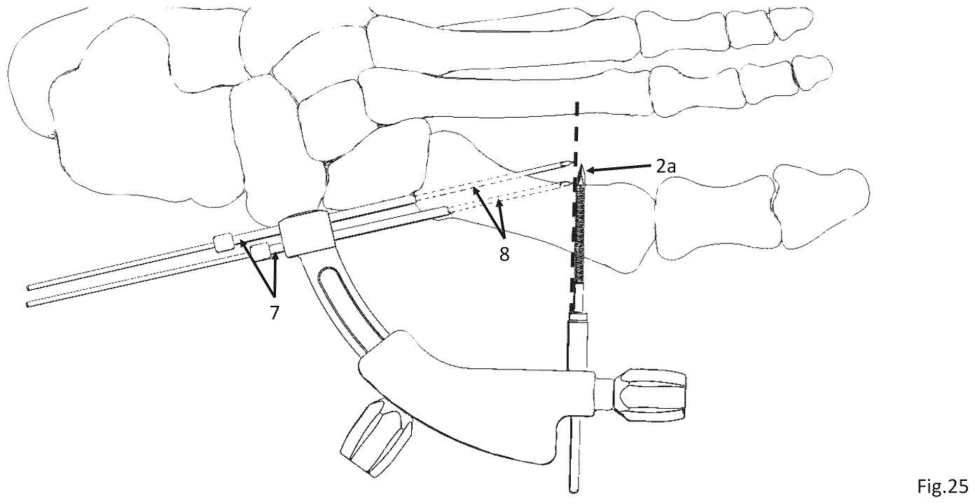

Referring to FIG. 25, the method can comprise a next step of inserting guiding wires 8 into the sleeves 7 and a part for example a second part of the first metatarsal bone until the guiding wires tips 8a reach the targeting wire 2 trocar tip 2a area, but such that the guiding wire tips does not come into contact with the targeting wire to give enough space for the cutting device (for example a burr) which will perform the osteotomy cut.

The method can comprise a next step of removing the sleeves 7, the targeting arm 4 and the guide body 3 while leaving the targeting wire and guide wires in place. The method comprises for example removing the sleeves from the targeting arm 4, for example by sliding the sleeves with respect to the guiding wires. The method can comprise a next step of retracting the targeting arm 4 in the guide body 3, this step comprises unscrewing the second clamping knob 6 so as to allow the targeting arm 4 to be movable with respect to the guide body 3. The targeting arm 4 can be removed from the guide wires 8 thanks to the internal and lateral grooves 4c, 4e of the targeting arm 4 which allow a passage of the guide wires 8. The method can comprise a next step of removing the guide body 3 from the targeting wire 2, this step comprises unscrewing the second clamping knob 6 and then pulling on the guide body 3.

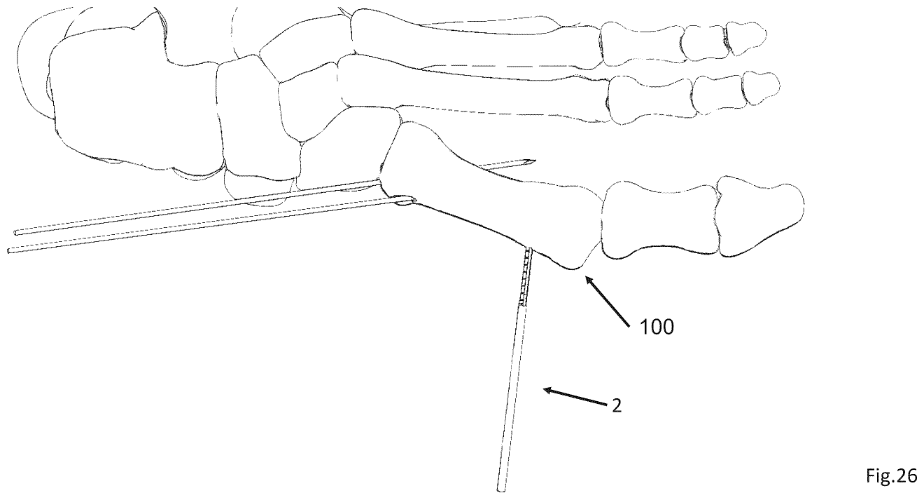

Referring to FIG. 26, the method can comprise a next step of removing the targeting wire 2 from the bone and replace it, at the same position and orientation by a cutting device, for example a burr allowing the burr to perform the cut of the first metatarsal head.

The method can comprise a next step of cutting the first metatarsal by means of the burr so as to form two bone fragments 102, 104. The cut forms a first part (the metatarsal head 102) and the second part (rest of the metatarsal 104) of the bone.

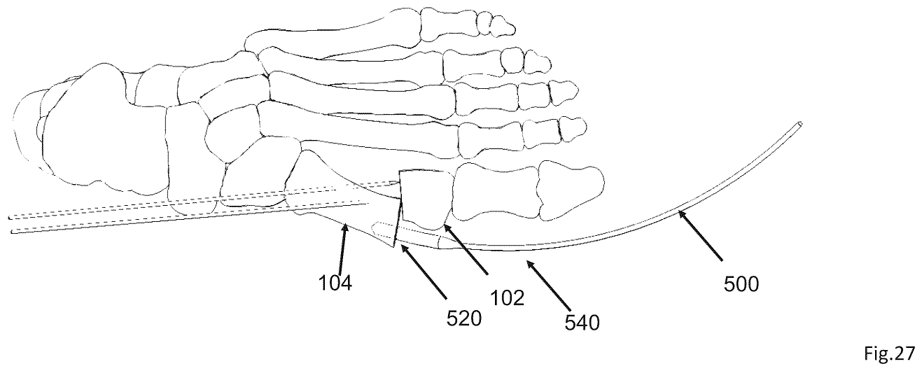

Referring to FIG. 27 the method can comprise a next step of translating the first metatarsal head 102, for example using a reduction tool 500, to translate the metatarsal head 102 until reaching the desired position. The reduction tool 500 can comprise a first part 520 configured to be inserted into the second part of the bone, and a second part 540 configured to form a lever.

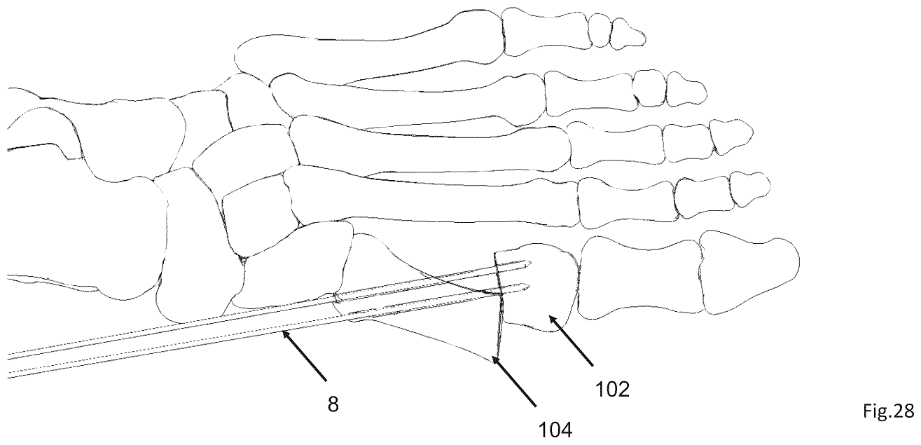

Referring to FIG. 28 the method can comprise a next step of pursuing the insertion of the guiding wire 8 until the guiding wire tips are inserted in the metatarsal head 102.

Referring to FIG. 29 the method can comprise a next step of inserting final implants, for example screws 300 into the bone fragments using associated instrumentation and the guiding wires.

The guiding wires 8 can be removed at this step, and the surgeon can finish the surgery by shaving down the medial prominence 400 until it is no longer palpable outside the skin.

The present invention provides a new surgical method, minimally invasive fixation of the guide on the metatarsal, stability of the orthopedic surgical guide, precise position of the guiding wires guiding the definitive implants, easy positioning of the screws with the help of the targeting arm, radiolucent instrumentation.

It shall be emphasized that, regardless of the embodiment of the surgical guide according to the invention, the latter enables an accurate insertion of the first proximal wire and that, regardless of the anatomy of the first metatarsal, an insertion of the second distal wire parallel to the first proximal wire, without any risk of interference between the screws, a symmetrical use of the surgical guide according to the invention and a reduced use of radiographic images.

Finally, it is quite obvious that the examples that have just been provided are only particular illustrations that are not restrictive with regards to the fields of application of the invention.

That which is claimed is:

1. A surgical guide for osteosynthesis surgery comprising:
a guide body comprising a head and a base, the head extending from one end of the base;
a guiding portion allowing the surgical guide to be positioned on a targeting wire inserted into a bone;
a locking device configured to secure the surgical guide on the targeting wire when the surgical guide is positioned on the targeting wire; and
a targeting arm adapted to cooperate with the base of the guide body to be displaceable relative to the guide body, the targeting arm having at a distal end thereof at least one opening for supporting the at least one guiding wire.

2. The surgical guide according to claim 1, wherein the guiding portion includes a through hole formed within the head of the guide body and a sleeve provided with a through hole, the sleeve extending from the head so that the through hole of the sleeve is aligned with the through hole of the head.

3. The surgical guide according to claim 1, wherein the locking device comprises a clamping knob comprising a threaded portion, and a threaded hole formed within the head of the guide body allowing to screw the clamping knob to secure the surgical guide to the targeting wire.

4. The surgical guide according to claim 1, further comprising an external visualization device configured to allow a preview of the at least one guiding wire insertion direction, the external visualization device comprising a connecting device configured to connect the external visualization device to the targeting arm.

5. The surgical guide according to claim 4, wherein the external visualization device comprises a first channel configured to hold the at least one guiding wire, wherein the connecting device comprises a first guiding pin receivable in the hole of the targeting arm, wherein the first channel and the first guiding pin are arranged on a same plane with an offset distance between the first channel and the first guiding pin.

6. The surgical guide according to claim 1, further comprising a monoaxial guiding device configured to hold a single guiding wire, the monoaxial guiding device comprising a single hole to hold the single guiding wire and further comprising a connecting device to connect the monoaxial guiding device to the targeting arm.

7. The surgical guide according to claim 1 wherein the guide body and the targeting arm are made of a radiolucent material.

8. An assembly, comprising
a surgical guide for osteosynthesis surgery comprising:
a guide body comprising a head and a base, the head extending from one end of the base;
a guiding portion allowing the surgical guide to be positioned on a targeting wire inserted into a bone;
a locking device configured to secure the surgical guide on the targeting wire when the surgical guide is positioned on the targeting wire; and
a targeting arm adapted to cooperate with the base of the guide body to be displaceable relative to the guide body, the targeting arm having at a distal end thereof at least one opening for supporting the at least one guiding wire; and
a targeting wire receivable through the hole of the head of the guide body.

9. The assembly according to claim 8, wherein the targeting wire comprises a first part configured to be inserted into the bone and a second part configured to hold the surgical guide, wherein the first part comprises a threaded portion.

10. The assembly according to claim 8, wherein the targeting wire comprises a physical stop configured to prevent the surgical guide from moving beyond the second part of the targeting wire.

11. A method comprising:
inserting a targeting wire in a bone;
positioning a surgical guide on the targeting wire;
inserting at least one guiding wire in the bone using the surgical guide;
performing a cut of the bone to form a first part of the bone and a second part of the bone;
translating the first part of the bone with respect to the second part of the bone; and
screwing at least one fixation screw into the first and second parts of the bone by using the at least one guiding wire.

12. The method according to claim 11, further comprising fixing the surgical guide on the targeting wire before inserting the at least one guiding wire in the bone using the surgical guide.

13. The method according to claim 12, wherein fixing the surgical guide on the targeting wire comprises screwing a clamping knob into a threaded hole of the surgical guide until a tip of the clamping knob comes into contact with the targeting wire.

14. The method according to claim 11, further comprising removing the surgical guide from the targeting wire and removing the targeting wire from the bone before performing the cut of the bone.

15. The method according to claim 11, wherein performing the cut of the bone comprises inserting a cutting device along the direction of insertion of the targeting wire.

16. The method according to claim 11, comprising previewing, by an external visualization device, the insertion direction of the at least one guiding wire, before inserting the at least one guiding wire in the bone.
